(19) United States
(12) Patent Application Publication
(10) Pub. No.: US 2012/0118293 A1
(43) Pub. Date: May 17, 2012

(54) Waugh stoma cover

(76) Inventor: Denice R. Taylor, Reynoldsburg, OH (US)

(21) Appl. No.: 12/927,396

(22) Filed: Nov. 15, 2010

(51) Int. Cl. A61M 16/00

(52) U.S. Cl. 128/205.29

(57) ABSTRACT

The Waugh Stoma Cover, consists of 2 Tefla band aids attached to each other by the tacky sides back to back, with nylon netting between the two. The nylon netting covers an opening in the two band aids approximately 1.″ in diameter.

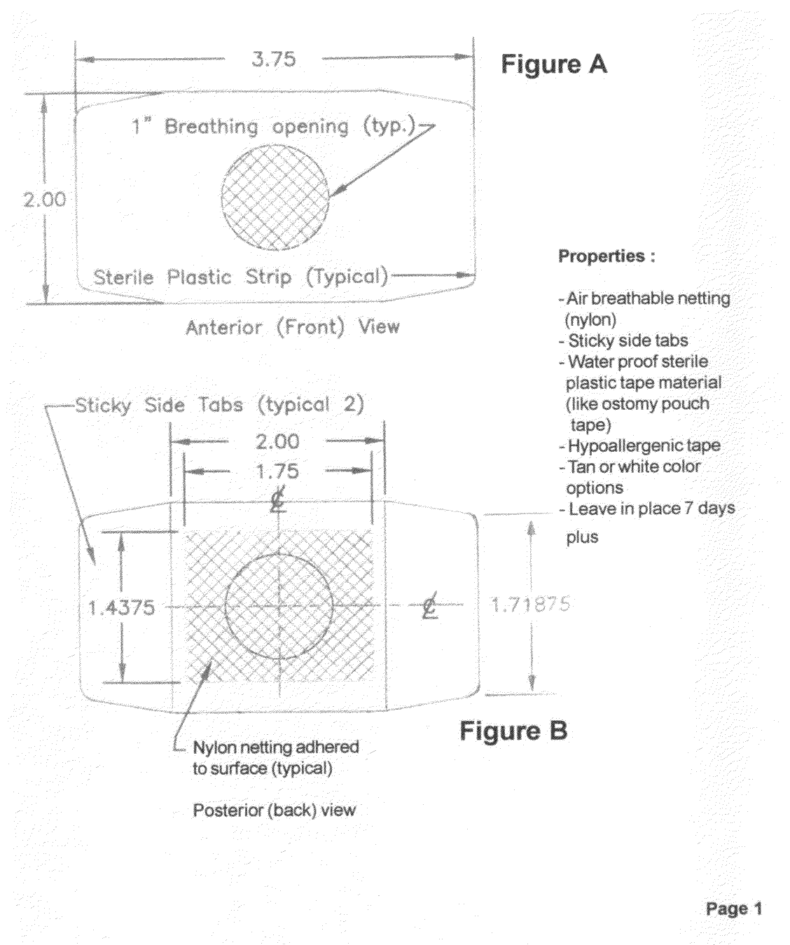

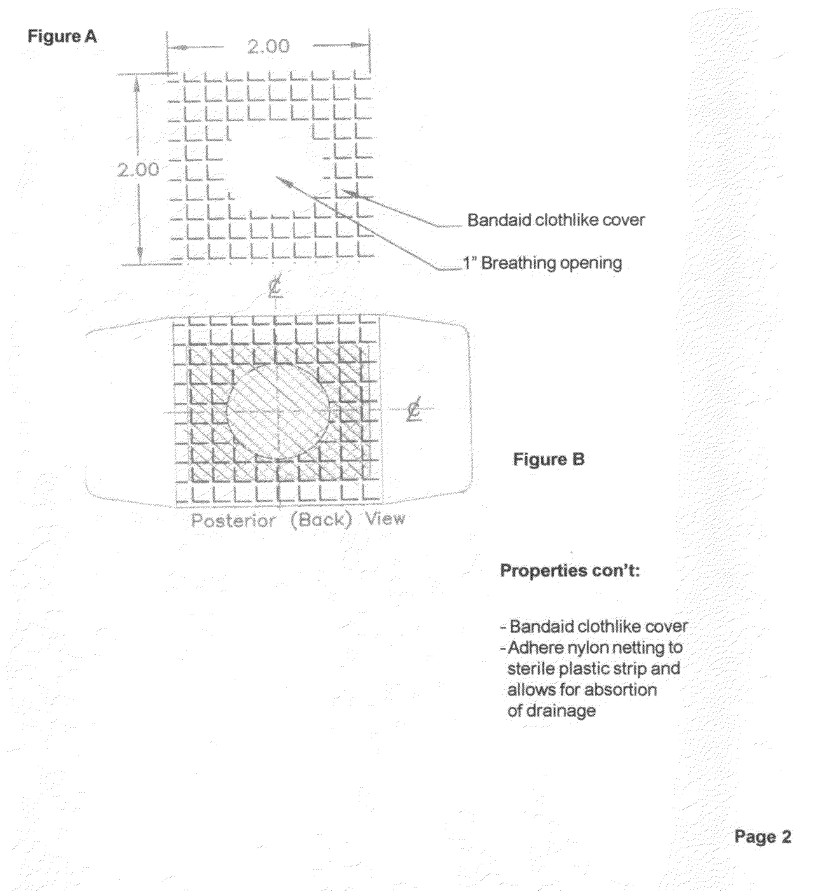

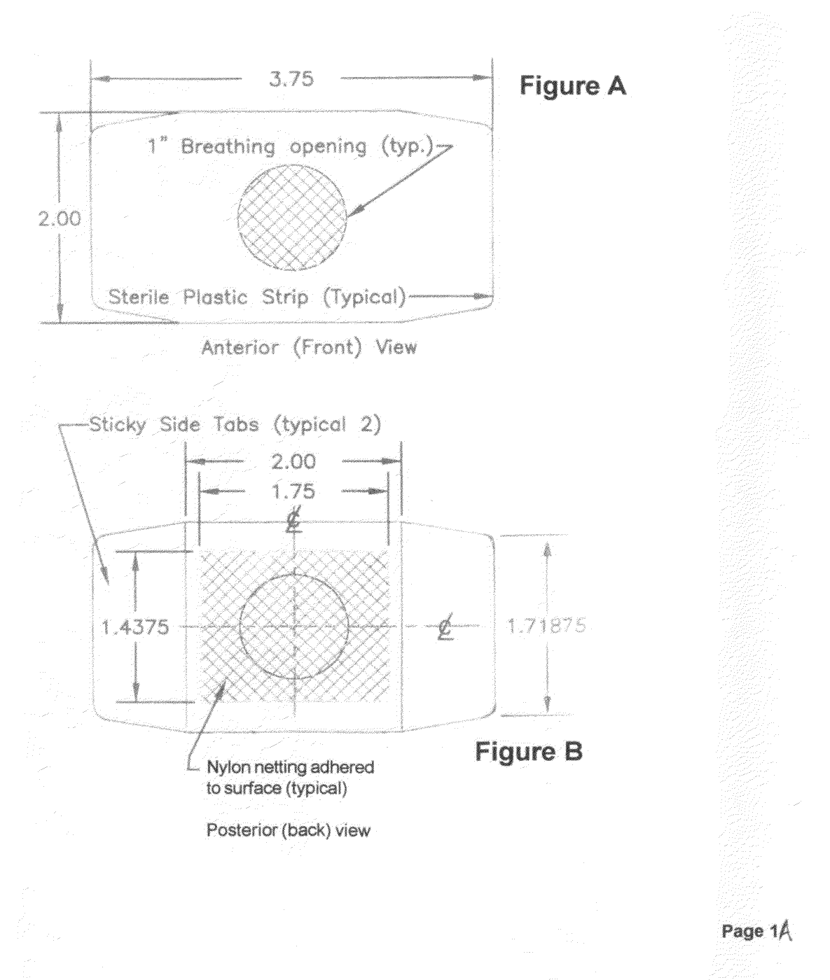

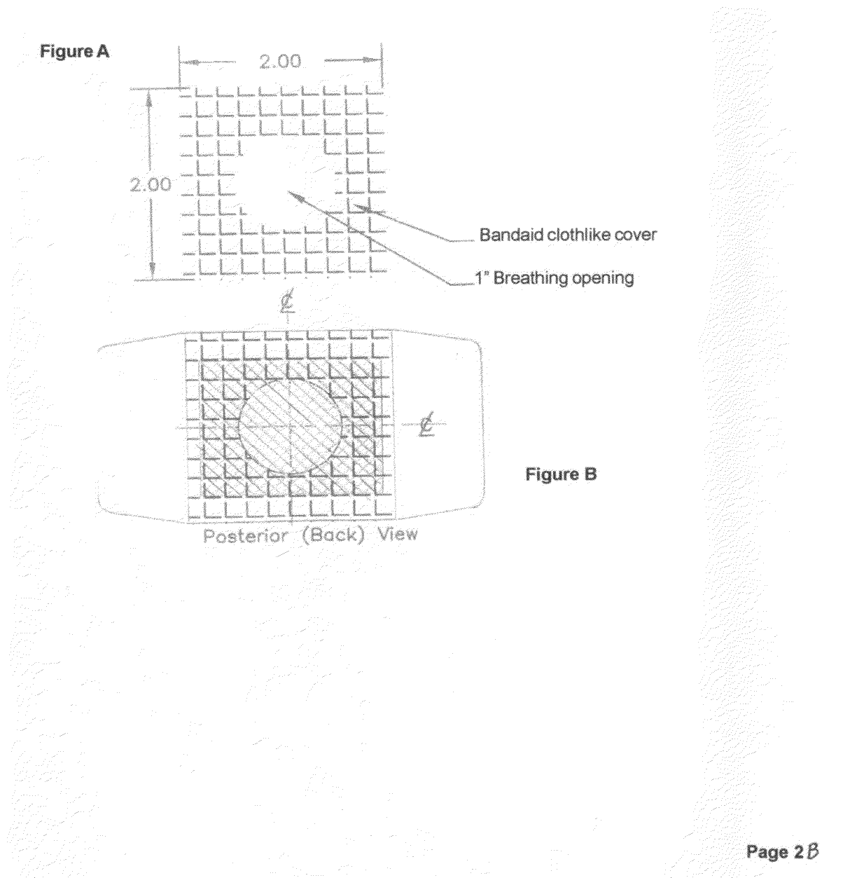

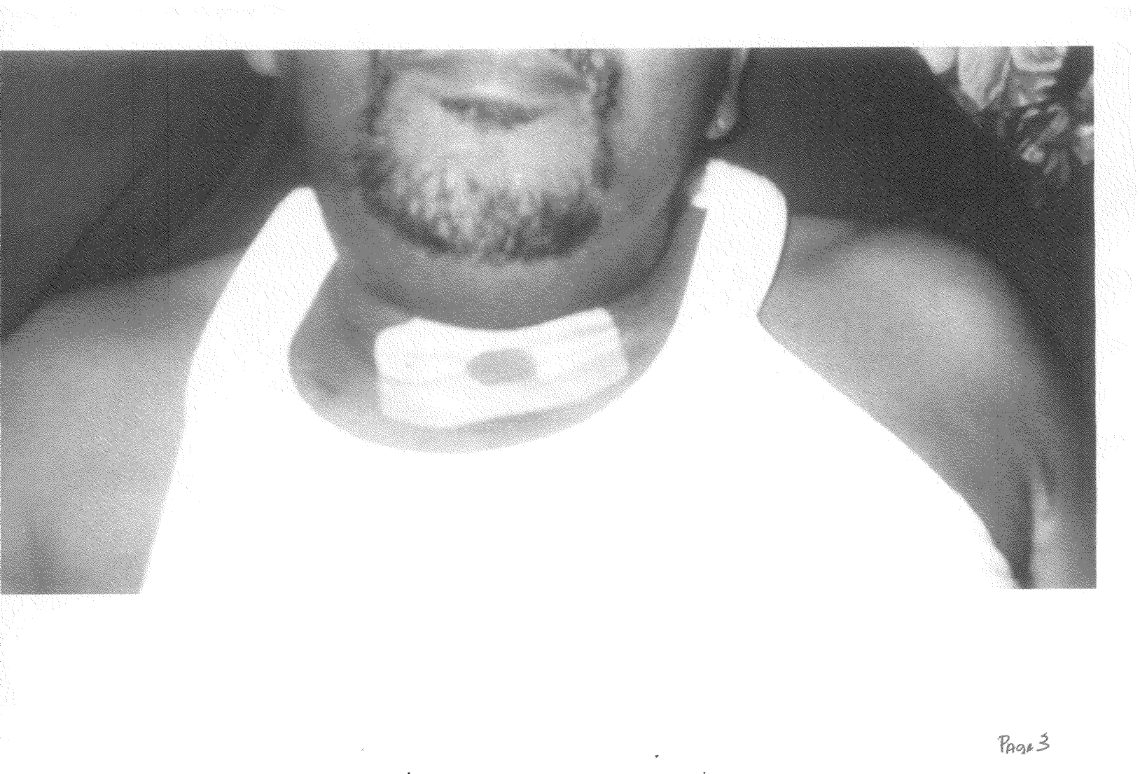

### BACKGROUND OR PROBLEM

People who have a laryngectomy, which is an ostomy opening in the throat area, suffer inconveniences and discomfort. The patient is susceptible to infection, discomfort, and the possibility of foreign objects entering or obstructing the opening. The patient is then at a serious health risk of infection, or worse death. Furthermore, in addition to the health risk involved, the patient is reluctant to interact with the public or participate in normal day-to-day activities because of the aesthetics and inconveniences of current laryngectomy products.

### SOLUTION

The Waugh Stoma Cover is specifically designed to alleviate the possibility of foreign objects entering the ostomy opening and it can protect the patient from harmful particles from entering the opening thus minimizing the risk of infection or any harmful objects from entering the body.

The Waugh Stoma Cover also gives the ostomy patient more mobility in day to day activities, in that The Waugh Stoma Cover can be worn comfortably with clothing without any cumbersome apparatuses needed.

### DETAILED DESCRIPTION OF INVENTION

The Waugh Stoma Cover are rectangular shaped opaque bandages with round openings in the center. Between the two bandages which are of two different sizes is a breathable nylon. The two bandages are attached together back to back with the two adhesive surfaces facing each other. Nylon netting is placed between the two adhesives surfaces. The inner surface of the bandage is attached to the patient's skin by the exposed adhesive surface of the larger outer bandage. The nylon netting covers a one inch opening that allows the passage of air from and to the patient without the hazards of objects entering in thru the opening on the patient's throat.

### BRIEF DESCRIPTIONS OF DRAWINGS ATTACHED

Page 1, FIGURE A shows The Waugh Stoma Front or sterile plastic tape that will be seen on a patient from the outside view. It shows the one inch opening which protects the patient and allows for breathing.

Page 1 Figure B shows the rear or underside of the sterile clothlike tape where a smaller sterile plastic tape is applied sticky side to sticky side to the front sterile tape. In between the two is the nylon netting. The nylon is air breathable.

Page 2, Figure A shows the actual measurements of the nylon netting that Covers the ostomy opening. It measures at 2 inches by 2 inches.

Page 2, Figure B shows the posterior view of the netting against the sterile tape.

Page 1A, Figure A shows the frontal view measurements of 3.75 inches×2 inches for the front sterile plastic tape with a 1 inch opening.

Page 1, Figure B shows the underside sticky side tabs and the nylon

Netting which is attached to the posterior.

Page 2B, Figure A and B shows the bandaid like cover over the 1 inch opening with the netting.

Page 3, Photo 1 of The Waugh Stoma Cover in actual use by a patient.

### BRIEF SUMMARY OF THE INVENTION

The Waugh Stoma Cover is a plastic/cloth/netting bandaid specifically designed to alleviate the possibility of foreign objects entering the ostomy opening and it can protect the patient from harmful particles from entering the opening thus minimizing the risk of infection or any harmful objects from entering the body.

The Waugh Stoma Cover also gives the ostomy patient more mobility in day to day activities, in that The Waugh Stoma Cover can be worn comfortably with clothing without any cumbersome apparatuses needed.

This product the “Waugh Stoma Cover” is a protective cover for tracheostomy patients who need a convenient yet safe wound care alternative to current tracheostomy products.

1. The Waugh Stoma Cover alleviates the possibility of foreign objects entering the ostomy opening.

2. The Waugh Stoma Cover minimizes the risk of infection.

3. More mobility and comfort for the patient.
